(19) United States
(12) Patent Application Publication
(10) Pub. No.: US 2025/0189422 A1
(43) Pub. Date: Jun. 12, 2025

(54) OPTICAL SENSOR, OPTICAL ARRANGEMENT AND METHOD FOR DETERMINING A REAL-TIME FLUID PROPERTY OF PARTICLES IN A TURBID MEDIUM

(71) Applicant: Agency for Science, Technology and Research, Singapore (SG)

(72) Inventors: Malini Olivo, Singapore (SG); Renzhe Bi, Singapore (SG); Amalina Attia, Singapore (SG); Gurpreet Singh, Singapore (SG); Ruochong Zhang, Singapore (SG)

(21) Appl. No.: 18/844,840

(22) PCT Filed: Mar. 6, 2023
(86) PCT No.: PCT/SG2023/050131

§ 371 Date: Sep. 6, 2024

(30) Foreign Application Priority Data
Mar. 7, 2022 (SG) 10202202241V

(51) Int. Cl. G01N 15/0205; G01N 15/0227; G01N 15/075; G01N 15/1434

(52) U.S. Cl. CPC G01N 15/0211; G01N 15/0227; G01N 15/075; G01N 15/1434

(57) ABSTRACT

According to embodiments of the present invention, an optical sensor is provided. The optical sensor includes a single piece of optical fiber including a modified fiber tip and a distal end opposite to the modified fiber tip. The distal end may be configured to optically communicate with an external light source and an external detector. The modified fiber tip may be configured to be positioned within a turbid medium to deliver light from the external light source to particles in the turbid medium, at least minimize the light being back reflected at the modified fiber tip and receive backscattered light from the particles for determining a real-time fluid property of the particles in the turbid medium by the external detector. According to further embodiments of the present invention, an optical apparatus, an optical arrangement, and a method for determining a real-time fluid property of the particles are also provided.

### CROSS-REFERENCE TO RELATED APPLICATION

This application claims the benefit of priority of Singapore patent application Ser. No. 10202202241V, filed 7 Mar. 2022, the content of it being hereby incorporated by reference in its entirety for all purposes.

### TECHNICAL FIELD

Various embodiments relate to an optical sensor, an optical apparatus and an optical arrangement, each of which includes the optical sensor, and a method for determining a real-time fluid property of particles in a turbid medium.

### BACKGROUND

For measurement of fluid property such as flow rate and molecular concentration inside tubes (e.g. blood vessel, pipeline, catheter), guide-wire-based sensing probes are typically used. For example, a Doppler guide wire has been widely used in clinical intravascular flow rate measurement. However, due to the limit of the size of ultrasound element, the smallest diameter the Doppler guide wire can reach is 0.014 inch (about 360 micron). This diameter may be good enough for bigger vessels but is not suitable for smaller arterials or venous. A smaller sensing probe is in demand.

Measurement of low level of turbidity of the scattering liquid medium requires high sensitivity. Fiber bragg grating (FBG) methods make use of a predetermined length of fiber for sensing purpose, where a longer length of grating has a higher sensitivity. However, low sensitivity challenges are still being faced in many applications because FBG sensing methods rely on the vibration of the fiber itself that may not be desirable for the applications.

Fiber optic technology presents a dynamic and customizable technology with absolute measurement readouts, stability to electromagnetic interference, excellent resolution and range, portability, multiplex possibility and economical in value. As such, optical fiber sensors prove to be promising techniques in remotely and continuously monitoring scattering particles concentration and size in flowing media.

However, there is still a need for a method and/or system that can achieve high measurement sensitivity within a significantly small probe size, while addressing at least the problems mentioned above.

### SUMMARY

According to an embodiment, an optical sensor is provided. The optical sensor may include a single piece of optical fiber including a modified fiber tip and a distal end, the distal end being opposite to the modified fiber tip, wherein the distal end may be configured to optically communicate with an external light source and an external detector; and the modified fiber tip may be configured to be positioned within a turbid medium to deliver light from the external light source to particles in the turbid medium, at least minimize the light being back reflected at the modified fiber tip, and receive backscattered light from the particles for determining a real-time fluid property of the particles in the turbid medium by the external detector.

According to an embodiment, an optical apparatus is provided. The optical apparatus may include the optical sensor according to an embodiment; and an optical circulator including a first port configured to optically couple with an external light source, a second port configured to optically couple with the optical sensor, and a third port configured to optically couple with an external detector.

According to an embodiment, an optical arrangement is provided. The optical arrangement may include the optical sensor according to an embodiment; a light source configured to emit light towards particles in a turbid medium through the optical sensor; a detector configured to receive backscattered light from the particles through the optical sensor for determining a real-time fluid property of the particles in the turbid medium; and an optical circulator including a first port, optically coupled with the light source, for receiving the light being emitted by the light source; a second port, optically coupled with the optical sensor, for directing the received light to the particles and receiving the backscattered light from the particles; and a third port, optically coupled with the detector, for directing the received backscattered light to the detector.

According to an embodiment, a method for determining a real-time fluid property of particles in a turbid medium is provided. The method may include providing a single piece of optical fiber including a modified fiber tip and a distal end opposite to the modified fiber tip, with the modified fiber tip positioned within a turbid medium, and the distal end in optical communication with a light source and a detector; delivering light, emitted by the light source, through the single piece of optical fiber to particles in the turbid medium; receiving, by the detector, backscattered light from the particles through the single piece of optical fiber, while at least minimizing the light being back reflected at the modified fiber tip; and processing the received backscattered light to determine the real-time fluid property of the particles.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1A shows a schematic view of an optical sensor, according to various embodiments.

FIG. 1B shows a schematic view of an optical apparatus, according to various embodiments.

FIG. 1C shows a schematic view of an optical arrangement, according to various embodiments.

FIG. 1D shows a flow chart illustrating a method for determining a real-time fluid property of particles in a turbid medium, according to various embodiments.

FIG. 2A shows a schematic cross-sectional side view of a modified fiber tip having a symmetrical needle shape, according to one example.

FIG. 2B shows a schematic cross-sectional side view of a modified fiber tip having an asymmetrical needle shape, according to another example.

FIG. 3 shows a schematic view illustrating a diffuse optical sensor and its setup in scattering fluid media or scattering liquid, according to various embodiments.

FIG. 4A shows a schematic cross-sectional side view depicting a flat fiber tip of an optical fiber probe where back reflection of light from the flat fiber tip is illustrated, according to one example.

FIG. 4B shows a schematic cross-sectional side view depicting an angle-polished flat fiber tip whereby back reflected light from the angle-polished flat fiber tip leaves the optical fiber probe through the fiber sidewall, according to one embodiment.

FIG. 5 shows a schematic cross-sectional side view depicting the angle-polished flat fiber tip of FIG. 4B in scattering media for detection of scattered light caused by the suspended particles present in the media of interrogation, according to one embodiment.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the phrase “at least substantially” may include “exactly” and a reasonable variance.

In the context of various embodiments, the term “about” as applied to a numeric value encompasses the exact value and a reasonable variance.

As used herein, the term “and/or” includes any and all combinations of one or more of the associated listed items.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

As used herein, the expression “configured to” may mean “constructed to” or “arranged to”.

Various embodiments provide multifunction measurement in scattering fluid media using one-piece optical fiber. More specifically, a method for multifunction sensing using only a single piece of optical fiber, that may be as small as about 100 micron in diameter, may be provided. The method may use the interaction between photons and the scattering particles, and not the vibration of fibers as employed in FBG sensing methods, to attain high sensitivity to the flow of the scattering particles such as red blood cells. To maintain high measurement sensitivity within a significantly small probe size, a unique design is adopted where the fiber tip of the single piece of optical fiber may be processed in special ways, and special signal processing methods may be utilized.

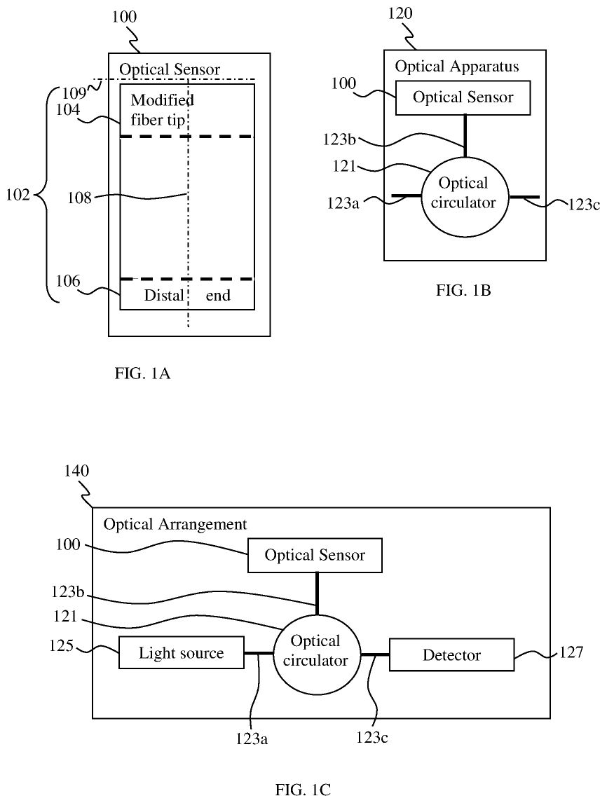

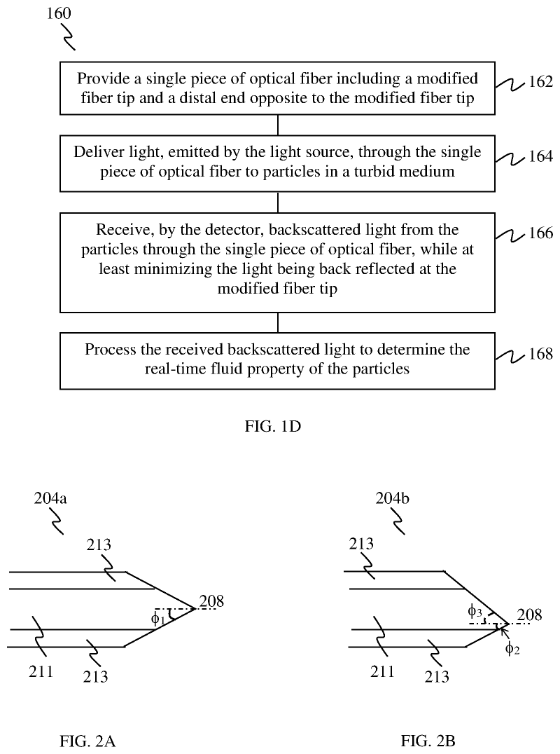

FIG. 1A shows a schematic view of an optical sensor 100, according to various embodiments. As seen in FIG. 1A, the optical sensor 100 includes a single piece of optical fiber 102 including a modified fiber tip 104 and a distal end 106, the distal end 106 being opposite to the modified fiber tip 104. The distal end 106 may be configured to optically communicate with an external light source (e.g. 125 in FIG. 1C) and an external detector (e.g. 127 in FIG. 1C). The modified fiber tip 104 may be configured to be positioned within a turbid medium (not shown in FIG. 1A) to deliver light from the external light source to particles in the turbid medium, at least minimize the light being back reflected at the modified fiber tip 104, and receive backscattered light from the particles for determining a real-time fluid property of the particles in the turbid medium by the external detector.

In the context of various embodiments, the expression “single piece of optical fiber” means only a sole piece of optical fiber. The term “modified” in relation to the fiber tip may refer to a fiber tip being pre-processed or engineered to cause a structural difference as compared to a flat, straight fiber end surface. A flat, straight fiber end surface is where the core and cladding are substantially flushed with each other along a same plane that is substantially perpendicular to a central axis of the optical fiber when the optical fiber is laid out along a straight line.

In other words, a single piece of optical fiber may be used as a probe for performing optical measurements in fluids of a turbid nature (monophasic fluids such as comprising scattering particles in suspension, amongst others). The turbid medium may be interchangeably referred to as a scattering medium or a liquid medium. The single piece of optical fiber may be used for both transmitting light to the particles and receiving light scattered or reflected by the particles. A measuring method for real-time flow rate and concentration of the scattering particles, by way of fiber optic elements including a remote optical circulator in a system of optical fiber cables and optical fiber sensors, may also be provided. The fiber probe tip, or interchangeably referred to a fiber tip, may be engineered to reduce or even eliminate the direct back scattering/reflection of the light transmitted, thereby increasing the sensitivity of measurements and increasing signal-to-noise ratio of the measurements. The direct back scattering/reflection of the light transmitted occurs at the fiber tip, more precisely, at the fiber end surface or fiber interface. The technique may make it possible in particular to characterize scattering and flowing fluid media while finding applications in various fields, such as intravascular blood flow and oxygenation sensor (biomedical), turbidity sensor in flowing water in pipes (environmental) and for monitoring cell growth (biotechnology), amongst others.

In various embodiments, the modified fiber tip 104 may be tapered at an angle with respect to a vertical axis 109 of the single piece of optical fiber 102, the angle being outside an acceptance angle of the single piece of optical fiber 102, and the vertical axis 109 being perpendicular to a central axis 108 of the single piece of optical fiber 102. The acceptance angle for an optical fiber, in this case being the single piece of optical fiber 102, is defined as the maximum angle of incidence at the interface of medium and a core of the optical fiber for which light ray enters and travel along the optical fiber. For example, the angle of the modified fiber tip 104 may be more than 15°. In other words, the modified fiber tip 104 may be sloped at less than or equal to 75° when measured from the vertical axis 109.

In other embodiments, the modified fiber tip 104 may include an anti-reflection coating. The anti-reflection coating may include a dielectric thin-film coating. In some examples, the anti-reflection coating may be a single anti-reflection coating, or a dual anti-reflection coating, or a broadband anti-reflection coating.

In yet other embodiments, the modified fiber tip 104 may be of a needle shape with a part of a core (e.g. 211) of the single piece of optical fiber 102 without an outer surrounding cladding (e.g. 213) of the single piece of optical fiber 102. The modified fiber tip 104 may be of a substantially symmetrical needle shape, as shown in a schematic cross-sectional side view of one example 204a in FIG. 2A. Each side of the substantially symmetrical needle shape may be sloped from the core 211 outwardly towards the cladding 213 at $1 being between 10° to 75° with respect the central axis 208. For example, 01 may be about 45°. The modified fiber tip 104 may be of an asymmetrical needle shape, as shown in a schematic cross-sectional side view of another example 204b in FIG. 2B. The asymmetrical needle shape may be sloped on each side with $2 and $3 being the same or different, each between 10° to 75° with respect the central axis 208.

In various embodiments, a sensing platform may be based on the single piece of optical fiber 102. The single piece of optical fiber 102 may have a core refractive index and a cladding refractive index, wherein the core refractive index being higher than the cladding refractive index. For example, the core refractive index may be in a range of 1.4 to 1.7, and the cladding refractive index may be in a range of 1.2 to 1.4, not inclusive of 1.4.

The single piece of optical fiber 102 may be a single-mode optical fiber or a multi-mode optical fiber.

FIG. 1B shows a schematic view of an optical apparatus 120, according to various embodiments. As seen in FIG. 1B, the optical apparatus 120 includes an optical sensor 100 according to various embodiments; and an optical circulator 121 including a first port 123a configured to optically couple with an external light source (not shown in FIG. 1B); a second port 123b configured to optically couple with the optical sensor 100, and a third port 123c configured to optically couple with an external detector (not shown in FIG. 1B). In this case, the optical circulator 121 is a three-port device designed such that the light from the external light source enters from the first port 123a and exits the second port 123b sending the light to the optical sensor 100, and the backscattered light received by the optical sensor 100 enters the second port 123b and exits the third port 123c sending the backscattered light to the external detector.

The optical apparatus 120 may include the same or like elements or components as those of the optical sensor 100 of FIG. 1A, and as such, the same numerals are assigned and the like elements may be as described in the context of the optical sensor 100 of FIG. 1A, and therefore the corresponding descriptions are omitted here.

FIG. 1C shows a schematic view of an optical arrangement 140, according to various embodiments. As seen in FIG. 1C, the optical arrangement 140 includes an optical sensor 100 according to various embodiments; a light source 125 configured to emit light towards particles in a turbid medium (not shown in FIG. 1C) through the optical sensor 100; a detector 127 configured to receive backscattered light from the particles through the optical sensor 100 for determining a real-time fluid property of the particles in the turbid medium; and an optical circulator 121. The optical circulator 121 may include a first port 123a, optically coupled with the light source 125, for receiving the light being emitted by the light source 125; a second port 123b, optically coupled with the optical sensor 100, for directing the received light to the particles and receiving the backscattered light from the particles; and a third port 123c, optically coupled with the detector 127, for directing the received backscattered light to the detector 127.

The optical arrangement 140 may include the same or like elements or components as those of the optical sensor 100 of FIG. 1A or the optical apparatus 120 of FIG. 1B, and as such, the same numerals are assigned and the like elements may be as described in the context of the optical sensor 100 of FIG. 1A or the optical apparatus 120 of FIG. 1B, and therefore the corresponding descriptions are omitted here.

In various embodiments, the light source 125 may include a coherent light source. For example, the light source 125 may be a laser. For example, the laser may have a power ranging from 0.01 mW to 5 mW, and a wavelength ranging from 400 nm to 1200 nm.

The detector 127 may include a pixeled camera, for example, a charged-coupled device (CCD) camera or a complimentary metal-oxide-semiconductor (CMOS) camera.

FIG. 1D shows a flow chart illustrating a method 160 for determining a real-time fluid property of particles in a turbid medium, according to various embodiments. At Step 162, a single piece of optical fiber (e.g. 102 in FIG. 1A) including a modified fiber tip (e.g. 104 in FIG. 1A) and a distal end (e.g. 106 in FIG. 1A) opposite to the modified fiber tip may be provided, with the modified fiber tip positioned within a turbid medium, and the distal end in optical communication with a light source (e.g. 125 in FIG. 1C) and a detector (e.g. 127 in FIG. 1C). At Step 164, light, emitted by the light source, may be delivered through the single piece of optical fiber to particles in the turbid medium. At Step 166, backscattered light from the particles may be received by the detector through the single piece of optical fiber, while the light being back reflected at the modified fiber tip may be at least minimized. At Step 168, the received backscattered light may be processed to determine the real-time fluid property of the particles.

In other words, the method 160 may be an optical method using backscattered photons in scattering fluid media for multifunction detection (instead of using back reflected photons in existing systems). More specifically, the single piece of optical fiber may be used for multifunction sensing, where it is used for both light delivery and light collection through a fiber circulator (e.g. 121 in FIGS. 1B and 1C). Currently, a single piece of optical fiber is the smallest sensing probe (about 100 micron in diameter) that may be used for intravascular blood flow measurement or other intra-pipe/tube monitoring. The method 160 may involve the same or like elements or components as those of the optical sensor 100 of FIG. 1A or the optical apparatus 120 of FIG. 1B or the optical arrangement 140 of FIG. 1C, and as such, the corresponding descriptions may be omitted here.

In various embodiments, the modified fiber tip may include at least one of the following: an angle tapered with respect to a vertical axis (e.g. 109 in FIG. 1A) of the single piece of optical fiber, the angle being outside an acceptance angle of the single piece of optical fiber, for example, the angle of the modified fiber tip being more than 15°; an anti-reflection coating; or a needle shape with a part of a core of the single piece of optical fiber without an outer surrounding cladding of the single piece of optical fiber.

The real-time fluid property of the particles may include a concentration of the particles in the turbid medium, or a flow rate of the particles in the turbid medium, or a viscosity of the particles in the turbid medium, or an average size of the particles in the turbid medium, or a Brownian motion rate of the particles in the turbid medium. The absorption spectrum of the turbid medium, or the optical scattering spectrum of the turbid medium may also be determined.

In various embodiments, the step of delivering the light emitted by the light source at Step 164 may include delivering the light via a first port (e.g. 123a in FIG. 1C) of an optical circulator (e.g. 121 in FIG. 1C) and directing the light to the particles in the turbid medium via a second port (e.g. 123b in FIG. 1C) of the optical circulator, the second port being optically coupled with the distal end of the single piece of optical fiber.

The step of receiving the backscattered light from the particles at Step 166 may include receiving the backscattered light from the particles via the second port and directing the received backscattered light to the detector via a third port (e.g. 123c in FIG. 1C) of the optical circulator.

While the method described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

Examples of a multifunction measuring method of scattering fluid media, such as flow of scattering liquid, concentration of scattered particles and viscosity, by the way of an optical fiber circulator system with a specially engineered tip will be described below.

The multifunction measuring method may be described in similar context to the method 160 of FIG. 1D, while the optical fiber circulator system and the specially engineered tip may include the same or like elements or components as those of the optical arrangement 140 of FIG. 1C and the modified fiber tip 104 in FIG. 1A, respectively, and as such, the similar ending numerals are assigned and the like elements may be as described in the context of the the optical arrangement 140 of FIG. 1C and the modified fiber tip 104 of FIG. 1A, respectively.

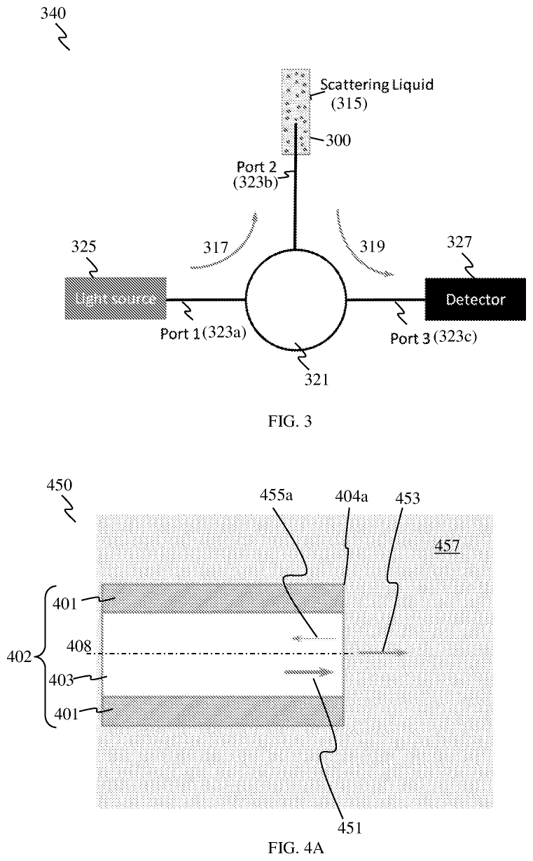

FIG. 3 shows a schematic view illustrating a diffuse optical sensor 300 and its setup 340 (involving the optical fiber circulator system) in scattering fluid media or scattering liquid 315, according to one example. The diffuse optical sensor 300 and the optical fiber circulator system may be described in similar context to the optical sensor 100 of FIG. 1A and the optical arrangement 140 of FIG. 1C, respectively.

The setup 340 includes a multi-mode optical fiber circulator 321. A light source 325 is optically coupled to port 1 323a. From port 1 323a, light travels to port 2 323b as indicated by a directional arrow 317, then from port 2 323b to port 3 323c as indicated by another directional arrow 319. Light cannot travel from port 1 323a to port 3 323c directly, by passing port 2 323b. In other words, the light source 325 sends a light signal through port 1 323a, which is then directed to port 2 323b. The light signal is scattered by the particles in the scattering liquid 315 as backscattered light that is sent through port 2 323b and then directed to port 3 323c. The port 2 323b of the circulator 321 is connected to the single piece of optical fiber 300 and port 3 323c is attached or optically coupled to a detector 327.

The light source 325 may be a coherent light source, such as lasers for illumination through port 1 323a. At the detector side, a grinding pattern (also known as speckle pattern) may be captured by the detector 327, which may include a pixelized detector, such as a CCD camera or CMOS camera. Through the analysis of the captured images from the detector 327, information such as flow rate, molecular concentration and viscosity may be extracted.

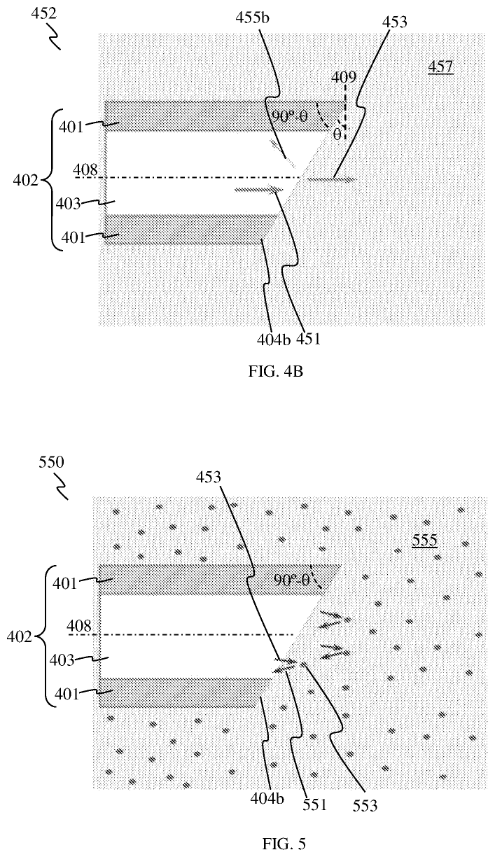

The working principles between an exemplary flat fiber tip (that is, with a flat, straight fiber end surface) and a specially engineered tip, described in similar context to the modified fiber tip 104 of FIG. 1A according to one embodiment, will be described with reference to FIGS. 4A, 4B and 5.

FIG. 4A shows a schematic cross-sectional side view 450 depicting a flat fiber tip 404a of an optical fiber probe 402 where back reflection of light 455a from the flat fiber tip 404a is illustrated, according to one example. The flat fiber tip 404a essentially lies on a plane that is substantially perpendicular to a central axis 408 of the optical fiber probe 402.

As seen in FIG. 4A, the optical fiber probe 402 in contact with the media 457 includes the core 403 and the outer surrounding cladding 401. The core 403 is the light-transmitting component of the optical fiber probe 402 with, for example, a refraction index of 1.51 (n₁, that being the core refractive index) while the outer surrounding cladding 408 has a relatively lower index of refraction, e.g. 1.33 (n₂, that being the cladding refractive index). This refraction index difference may ensure light transmission through the core 408 via total internal reflection. Other refraction index differences may also be possible to facilitate total internal reflection. In other words, the light 451 sent through the core 408 experiences, at the flat fiber tip 404a that is the fiber interface, a portion of light 453 being transmitted into the media 457 and another portion of light 455a being reflected back into the core 403. At the flat fiber tip 404a, some of the light propagating inside the optical fiber probe 402 reflects back from the flat fiber tip 404a reducing the signal-to-noise of the measurements. Some existing systems may make measurements of the media using this working principle.

In contrast, FIG. 4B shows a schematic cross-sectional side view 452 depicting the specially engineered tip, here being an angle-polished flat fiber tip 404b, whereby back reflected light 455b from the angle-polished flat fiber tip 404b leaves the optical fiber probe 402 through the fiber sidewall which may include the cladding 401, according to one embodiment. The angle-polished flat fiber tip 404b, or interchangeably referred to as an angled fiber tip or a bevelled fiber end surface, may be described in similar context to the modified fiber tip 104 of FIG. 1A and essentially lies on a plane that is at an angle θ with respect to the vertical axis 409 of the optical fiber probe 402 (which may be described in similar context to the single piece of optical fiber 102 of FIG. 1A). The vertical axis 409 is perpendicular to the central axis 408 of the optical fiber probe 402.

By polishing the fiber end surface at a specific angle θ of more than 15° to give the angle-polished flat fiber tip 404b, the incidence of the angle-polished flat fiber tip 404b is modified, allowing the back reflected light 455b to leave the optical fiber probe 402 through the fiber sidewall that includes the cladding 401. In effect, the angle-polished flat fiber tip 404b, sloping at less than or equal to 75° (i.e. provided by 90°-θ as seen in FIG. 4B), redirects the back reflection at an angle outside of the fiber acceptance angle, ensuring no back reflection is transmitted down the core 403 (in contrast to that as shown by 455a in FIG. 4A). This is desirable as back reflection may reduce the signal by a significant percentage and in high power instances, may over-heat fibers at sharp bends or damage the light source.

FIG. 5 shows a schematic cross-sectional side view 550 depicting the angle-polished flat fiber tip 404b of FIG. 4B in scattering media 555 for detection of scattered light 551 caused by the suspended particles 553 present in the media 555 of interrogation, according to one embodiment.

When the optical fiber probe 402 is placed in the scattering fluid media 555 (which may be interchangeably be referred to as a turbid medium or a turbid media), light 453 is sent through the core 403 and outwardly from the angle-polished flat fiber tip 404b to the particles 553 of the scattering fluid media 555, the scattered (or backscattered) light 551 from the particles 553 in the media 555 provides the signal for determination of a fluid property of the media 555 (for example, the concentration of particles 553) without interference from back reflected light (not shown in FIG. 5) at the angle-polished flat fiber tip 404b of FIG. 4B.

Other approaches to eliminate or at least reduce direct back reflection may include a) the specially engineered tip involving an anti-reflection coating on the surface of the fiber tip, and/or b) the specially engineered tip involving polishing the fiber tip into a needle shape to expose the fiber core without cladding for a short length. Essentially, the anti-reflection coating may prevent any back reflected light at the fiber tip, and the needle-shaped fiber tip may work in a similar manner as the angle-polished flat fiber tip 404b of FIG. 4B.

As can be appreciated, the specially engineered tip provides a special fiber tip engineering patterns to reduce/eliminate back reflection.

Most existing fiber sensors use Fabry-Perot (F-P) configuration or fiber Bragg grating (FBG) methods, which produce interfere patterns or shift of the Bragg wavelength if the pressure/flow/temperature around the fiber tip change. Such existing fiber sensors do not use the direct interaction between photons and the scatterers in the media and the sensing functions are based on the stress/pressure/bending of the fiber tip itself. Various embodiments of this invention are not dependent on the physical status of the fiber itself, but using a single piece of fiber as a medium for both sending photons into the scattering liquid (media) and receiving the back scattered photons. Then the flow/concentration/viscosity information may be calculated based on the interaction between photons and scatterers/absorbers in the liquid. It should be understood and appreciated that the concept of various embodiments of the present invention is vastly different from most existing fiber sensing methods.

As discussed above, to achieve sending and receiving light through a single piece of optical fiber, a fiber circulator (e.g. 321 in FIG. 3) may be used. However, back reflection light 455a (as shown in FIG. 4A) from port 2 323b (shown in FIG. 3) may be a significant noise source because these photons do not enter the liquid media 457 but are directly reflected back at the fiber boundary (e.g. the core 408). Therefore, these back reflection photons do not carry information of the liquid media 457. The collection efficiency is significantly low for backscattered photons by the scatterers (scattering particles) because of the size limit of a single piece of optical fiber. Back reflection noise makes it substantially impossible to extract useful information. To address this challenge, several ways of engineering, as mentioned above, are proposed, namely, polishing the tip (e.g. 404b) at port 2 323b with an angle of more than 15° with respect to the vertical axis of the single piece of optical fiber, engineering a needle shape at port 2 323b, or coating an anti-reflection layer at the tip (e.g. 404a or 404b) at port 2 323b. Currently, polishing the tip (e.g. 404b) at port 2 323b with an angle of more than 15° with respect to the vertical axis of the single piece of optical fiber may be the easiest and cheapest processing method out of the three ways mentioned. It may be possible to combine two of the ways (e.g. polishing the polishing the tip at port 2 323b with an angle of more than 15° with respect to the vertical axis, and coating the angled tip). However, this adds on the cost and the efficiency may remain somewhat the same as using just one way.

In fiber communication, while angled polishing of fiber tip has been taught to reduce back reflection, such disclosures are limited to having fiber tips being polished at an angle of 5° to 15° and the most common angle may be about 8° with respect to the vertical axis. This is because an angle of more than 15° with respect to the vertical axis results in significant signal loss through fiber cladding in fiber communication, that results in low sensitivity. More than often, such smaller angled fiber tips may be employed in air, but not in a liquid (scattering or turbid medium).

In constrast, various embodiments of the present invention teach that a polishing angle of more than 15° with respect to the vertical axis has to be used for sensing the backscattered light. This large angle reduces or even eliminates the light being reflected at the fiber interface. This allows more light going into the liquid medium to interact with particles, then gets scattered back into the fiber. It is believed that experts in fiber communication relevant areas do not have sufficient knowledge in optical scattering theory in scattering media or laser coherence analysis, while experts in diffuse optics do not have sufficient knowledge in fiber tip engineering or fiber circulators. Deep knowledge and rich experimental experience in optical fiber, fiber tip engineering, fiber circulator, diffuse optics and laser coherence analysis, are advantageously required to provide the various embodiments of the present invention, therefore rendering the invention not trivial.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. An optical sensor comprising
a single piece of optical fiber comprising a modified fiber tip and a distal end, the distal end being opposite to the modified fiber tip, wherein
the distal end is configured to optically communicate with an external light source and an external detector; and
the modified fiber tip is configured to be positioned within a turbid medium to deliver light from the external light source to particles in the turbid medium, at least minimize the light being back reflected at the modified fiber tip, and receive backscattered light from the particles for determining a real-time fluid property of the particles in the turbid medium by the external detector.

2. The optical sensor as claimed in claim 1, wherein the modified fiber tip is tapered at an angle with respect to a vertical axis of the single piece of optical fiber, the angle being outside an acceptance angle of the single piece of optical fiber, and the vertical axis being perpendicular to a central axis of the single piece of optical fiber.

3. The optical sensor as claimed in claim 2, wherein the angle of the modified fiber tip is more than 15°.

4. The optical sensor as claimed in claim 1, wherein the modified fiber tip comprises an anti-reflection coating.

5. The optical sensor as claimed in claim 4, wherein the anti-reflection coating comprises a dielectric thin-film coating.

6. The optical sensor as claimed in claim 4 or 5, wherein the anti-reflection coating comprises a single anti-reflection coating, or a dual anti-reflection coating, or a broadband anti-reflection coating.

7. (canceled)

8. The optical sensor as claimed in claim 1, wherein the modified fiber tip is of a symmetrical needle shape.

9. The optical sensor as claimed in claim 1, wherein the modified fiber tip is of an asymmetrical needle shape.

10. The optical sensor as claimed in claim 1, wherein the single piece of optical fiber has a core refractive index and a cladding refractive index, the core refractive index being higher than the cladding refractive index.

11. An optical apparatus comprising:
an optical sensor as claimed in claim 1; and
an optical circulator comprising:
a first port configured to optically couple with an external light source,
a second port configured to optically couple with the optical sensor, and
a third port configured to optically couple with an external detector.

12. An optical arrangement comprising:
an optical sensor as claimed in claim 1;
a light source configured to emit light towards particles in a turbid medium through the optical sensor;
a detector configured to receive backscattered light from the particles through the optical sensor for determining a real-time fluid property of the particles in the turbid medium; and
an optical circulator comprising:
a first port, optically coupled with the light source, for receiving the light being emitted by the light source;
a second port, optically coupled with the optical sensor, for directing the received light to the particles and receiving the backscattered light from the particles; and
a third port, optically coupled with the detector, for directing the received backscattered light to the detector.

13. The optical arrangement as claimed in claim 12, wherein the light source comprises a coherent light source.

14. The optical arrangement as claimed in claim 12, wherein the light source is a laser.

15. The optical arrangement as claimed in claim 12, wherein the detector comprises a pixeled camera.

16. The optical arrangement as claimed in claim 12, wherein the detector comprises a charged-coupled device camera or a complimentary metal-oxide-semiconductor camera.

17. A method for determining a real-time fluid property of particles in a turbid medium, the method comprising:
providing a single piece of optical fiber comprising a modified fiber tip and a distal end opposite to the modified fiber tip, with the modified fiber tip positioned within a turbid medium, and the distal end in optical communication with a light source and a detector;
delivering light, emitted by the light source, through the single piece of optical fiber to particles in the turbid medium;
receiving, by the detector, backscattered light from the particles through the single piece of optical fiber, while at least minimizing the light being back reflected at the modified fiber tip; and
processing the received backscattered light to determine the real-time fluid property of the particles.

18. The method as claimed in claim 17, wherein the modified fiber tip comprises at least one of the following:
an angle tapered with respect to a vertical axis of the single piece of optical fiber, the angle being outside an acceptance angle of the single piece of optical fiber, and the vertical axis being perpendicular to a central axis of the single piece of optical fiber;
an anti-reflection coating; or
a needle shape with a part of a core of the single piece of optical fiber without an outer surrounding cladding of the single piece of optical fiber.

19. The method as claimed in claim 18, wherein the angle of the modified fiber tip is more than 15°.

20. The method as claimed in claim 17, wherein the real-time fluid property of the particles comprises a concentration of the particles in the turbid medium, or a flow rate of the particles in the turbid medium, or a viscosity of the particles in the turbid medium, or an average size of the particles in the turbid medium, or a Brownian motion rate of the particles in the turbid medium.

21. The method as claimed in claim 17,
wherein the step of delivering the light emitted by the light source comprises delivering the light via a first port of an optical circulator and directing the light to the particles in the turbid medium via a second port of the optical circulator, the second port being optically coupled with the distal end of the single piece of optical fiber; and
wherein the step of receiving the backscattered light from the particles comprises receiving the backscattered light from the particles via the second port and directing the received backscattered light to the detector via a third port of the optical circulator.
